(19) United States
(12) Patent Application Publication
(10) Pub. No.: US 2019/0330376 A1
(43) Pub. Date: Oct. 31, 2019

(54) PEPTIDES DERIVED FROM KININOGEN-1 FOR PROTEIN DRUGS IN VIVO HALF-LIFE EXTENSIONS

(71) Applicant: Development Center for Biotechnology, Taipei (TW)

(72) Inventors: Chen-Li Chien, Taipei (TW); Gregory Jiann Chen, Taipei (TW); Chuan-Lung Hsu, Taipei (TW); Jei-Hwa Yu, Taipei (TW); Hsien-Yu Tsai, Taipei (TW); Show-Shan Sheu, Taipei (TW); Wei-Jung Chang, Taipei (TW); Chia-Cheng Wu, Taipei (TW)

(73) Assignee: Development Center for Biotechnology, Taipei (TW)

(21) Appl. No.: 16/475,121

(22) PCT Filed: Dec. 29, 2017
(86) PCT No.: PCT/US2017/069134

§ 371 Date: Jun. 30, 2019

Related U.S. Application Data
(60) Provisional application No. 62/440,251, filed on Dec. 29, 2016.

(51) Int. Cl. C07K 16/46; C07K 14/745

(52) U.S. Cl. CPC C07K 16/468; C07K 14/745; A61K 2039/505; C07K 2317/94; C07K 2317/41; C07K 2317/31

(57) ABSTRACT

A recombinant protein drug includes a parent protein drug coupled with a modified kininogen-1 peptide. The modified kininogen-1 peptide has the sequence of SEQ ID NO:2 or a homolog having a sequence identity of 80% or higher. The parent protein drug is a bispecific antibody having a first targeting domain linked by a bridging domain with a second targeting domain. The modified kininogen-1 peptide is fused between the first targeting domain and the bridging domain, or between the bridging domain and the second targeting domain. A method for increasing the serum half-life of a protein drug includes constructing a fusion protein comprising the protein drug coupled with a modified kininogen-1 peptide.

### BACKGROUND OF INVENTION

### Field of the Invention

The present invention relates to methods for extending in vivo half-lives of protein drugs and protein drugs prepared by such methods.

### Background Art

Half-life extension of a protein drug (such as a bispecific antibody (bsAb) in a single-chain fragment variable (scFv) format) can decrease the need for repeated administrations and can increase in vivo efficacies. Several approaches are available to extend the in vivo half-lives of protein drugs, including attachments of polyethylene glycol (PEG), carbohydrates, or glycopeptides.

Attachments of a highly-glycosylated peptide may increase the hydrodynamic radius of a protein drug, thereby increasing its retention in serum. This approach has been shown to be effective in half-life extension of follicle stimulating hormones (FSH) by attaching a highly-glycosylated carboxyl terminal peptide (CTP) derived from human chorionic gonadotropin protein. The half-life extending ability of CTP on the FSH protein was further increased when multiple CTPs were ligated. (see U.S. Pat. No. 6,225,449).

While the prior art methods have been able to extend protein drugs in vivo half-lives, there is still a need for better methods that can extend the half-lives of protein drugs.

### SUMMARY OF THE INVENTION

Embodiments of the invention relate to methods for extending protein drugs in vivo half-lives and protein drugs having extended in vivo half-lives. In accordance with embodiments of the invention, a protein drug may be modified with a highly-glycosylated peptide to extend its in vivo half-life. Preferably, the highly-glycosylated peptide is derived from a native protein; more preferably, the highly-glycosylated peptide is derived from a blood plasma protein, such as kininogens.

In accordance with embodiments of the invention the highly-glycosylated peptides are derived from human kininogen-1 (KNG1). Particularly, a highly-glycosylated peptide may be a human KNG1-derivded peptide K07, which was generated by joining two highly-glycosylated regions from the KNG1 protein. The 71 amino acids long K07 peptide comprises at least two asparagine, three serine, and six threonine amino acids that have the strong preference for glycosylation (FIG. 2). This novel K07 peptide may be ligated to a protein drug (such as a bispecific antibody (bsAb)). Ligation of the protein drug to the heavily glycosylated K07 peptide can provide an enhanced serum retention time due to the increased hydrodynamic radius of the protein.

In accordance with embodiments of the invention, the protein drugs may be any biologically active peptides or proteins, such as antibodies (including bispecific antibodies, scFv, mAb, etc.). A bispecific antibody (bsAb) comprises two specific binding domains (e.g., variable domains or scFv) linked by a bridging domain.

An example of a bsAb may comprise an anti-Her2 scFv and an anti-CD3 scFv joined together by a kappa light chain and a linker (FIG. 3). Ligation of the scFv bsAb (#152) to a heavily glycosylated K07 peptide can increase its hydrodynamic radius (increased bulk), resulting in extended serum retention time.

In one aspect, embodiments of the invention relate to recombinant protein drugs. A recombinant protein drug in accordance with one embodiment of the invention includes a parent protein drug coupled with a modified kininogen-1 peptide. In accordance with some embodiments of the invention, the modified kininogen-1 peptide has the sequence of SEQ ID NO:2 or a homolog thereof with 80% or higher sequence identity. In accordance with some embodiments of the invention, the parent protein drug is a bispecific antibody having a first targeting domain linked by a bridging domain with a second targeting domain. In accordance with some embodiments of the invention, the modified kininogen-1 peptide is fused between the first targeting domain and the bridging domain, or between the bridging domain and the second targeting domain.

In one aspect of the invention relate to methods for increasing the serum half-life of a protein drug. A method in accordance with one embodiment of the invention includes constructing a fusion protein comprising the protein drug coupled with a modified kininogen-1 peptide.

Other aspects and advantages of the invention will be apparent from the following description and the appended claims.

### BRIEF DESCRIPTION OF THE DRAWINGS

FIG. 1 shows human kininogen-1 protein sequence (SEQ ID NO:1). Bold and underlines indicate the amino acids within the sequence that are highly glycosylated.

FIG. 2 shows a 71-amino acid long kininogen-1 spliced peptide (SEQ ID NO:2) in accordance with embodiments of the invention. The asparagine, serine, and threonine residues with higher potential for N-linked or O-linked oligosaccharide attachments are highlighted as bold and underlined.

FIG. 3 shows an example of a bispecific antibody with a kappa bridge linker and a K07 peptide in accordance with one embodiment of the invention. In this example, the bispecific antibody has an anti-Her2 domain at the N-terminus and an anti-CD3 domain at the C-terminus. The arrow indicates the location of insertion for the K07 peptide in this particular embodiment.

FIG. 4 shows results of analysis of bsAb constructs by SDS-PAGE. Lane 2 is the parental bsAb construct #152 and lane 6 with the higher molecular weight is the purified K07-bsAb construct with the K07 peptide insertion.

FIG. 5 shows ELISA profiles of K07-containing bsAb in binding analysis. Standard curves are generated for both the parental bsAb construct (#152) and the parental bsAb #152 with K07 peptide insertion (152-K07).

FIG. 6 shows a pharmacokinetics (PK) study of a K07-containing bsAb. The control antibody is shown as bsAb #152 without the K07 peptide insert. Serum samples were collected from mice before (pre-dose) and at 11 timepoints (ranging from 5 min to 192 hr) after the injection of the bsAb samples. Serum concentrations of the bsAbs were determined with ELISA and shown as-concentrations (ug/ml) vs. time (hours).

FIG. 7A and FIG. 7B show the results of collision-induced dissociation (CID) analysis (using mass spectrometer) of N-glycosylation sites in peptide fragments derived from the K07-containing bsAb. The antibody is bsAb #152 with a K07 peptide insert. The precursor ion is a doubly-charged ion with m/z at 1311.5 and 1093.4 individually. Figures show CID MS/MS analysis data of the precursor ion. The N-glycosylation sites were determined by CID, electron-transfer dissociation (ETD) annotation and oligosaccharide biosynthesis pathway. The circle represents Man (mannose), the square represents GlcNAc (N-acetylglucosamine), and the broken lines represent dissociation of glycopeptides.

FIG. 8 shows the results of electron-transfer dissociation (ETD) analysis of O-glycosylation sites in peptide fragments derived from the K07-containing bsAb. The antibody is bsAb #152 with a K07 peptide insert. The top panel illustrates representative ETD analysis data of positive ions derived from an O-glycosylated peptide. The series ions of C and Z demonstrated the GalNAc glycan attach to the AS peptide. The 5³²⁶ is the glycosylation site due to the knowledge of biosynthesis pathway. Summary of identified glycosylation sites (highlighted as bold and underlined) from ETD spectra of glycopeptides from #152-K07 protein are shown in the table.

FIG. 9 shows the results of SDS-PAGE analysis of the K07-containing bsAb with or without mutations at glycosylation sites. Non-reduced (left) and reduced (right) samples were prepared and ran in the gel. Protein 1 (lanes 1): the parental bsAb construct #152; Protein 2 (lanes 1): the bsAb #152 with the K07 peptide insertion; Protein 3 (lanes 3): the mutant form of bsAb #152-K07 by substituting alanine for eleven amino acid residues (which had been characterized as shown in FIGS. 7 and 8 and highlighted in FIG. 2) in the K07 peptide.

FIG. 10 shows a pharmacokinetics (PK) study of the K07-containing bsAb with or without mutations at the glycosylation sites. The control antibody is bsAb #152 without a K07 peptide insert. The bsAb #152-K07 11 mut is a mutant form derived from the bsAb #152-K07 by substituting alanine for eleven amino acid residues in the K07 peptide highlighted in FIG. 2. Serum samples were collected from mice before (pre-dose) and at 11 time points (ranging from 5 min to 192 hr) after the injection of the bsAb samples. Serum concentrations of the bsAbs were determined with ELISA and shown as concentrations (μg/mL) vs. time (hr) in the top panel. Summary of the PK study comparing the parental bsAb construct #152 and the K07 peptide-containing bsAb #152 with or without glycosylation site mutations is shown in the bottom panel.

### DETAILED DESCRIPTION

Embodiments of the invention relate to extension of in vivo half-lives of peptide or protein drugs based on novel highly-glycosylated peptides. The peptide or protein drugs may include antibodies (or binding fragments thereof), hormones, cytokines, etc. While embodiments of the invention encompass both peptide and protein drugs, for simplicity and clarity of description, the term “protein drug” will be used to include both peptide and protein drugs. In accordance with embodiments of the invention, an antibody may include a monoclonal antibody (mAb), a bispecific (or multispecific) antibody, an antibody-drug conjugate, etc. All these antibody variants may be referred to as “antibody” in this description. The highly-glycosylated peptides may be derived from a natural glycoprotein, preferably a serum protein, such as kininogen-1 (KNG1). The natural protein derivatives (especially, serum proteins) are less likely to induce undesirable immune responses. The extension of the in vivo half-lives of protein drugs is based on the principle of increased hydrodynamic radius of the protein.

In accordance with embodiments of the invention the highly-glycosylated peptides may be derived from human kininogen-1 (KNG1). For example, such highly-glycosylated peptides may be generated by splicing glycosylated fragments from KNG1. Based on this approach, a highly-glycosylated peptide K07 (SEQ ID NO:2; FIG. 2) was generated by joining two highly-glycosylated regions from the KNG1 protein. In this description, this K07 highly-glycosylated peptide will be used as an example to illustrate embodiments of the invention. However, one skilled in the art would appreciate that this is only for illustration and is not intended to limit the scope of the present invention because other modification and variations are possible without departing from the scope of the invention. Such variations or modifications, for example, may include amino-acid substitutions, additions, and/or deletions in K07 peptide. All these modified K07 peptides will be referred to as “homologs.”

In the particular example, K07 is a 71-amino acids long peptide that comprises at least two asparagines, three serines, and six threonines that have great potentials for glycosylations. This K07 peptide may be ligated to a protein drug (such as a bispecific antibody (bsAb)). Ligation may be accomplished by chemical means (e.g., cross-linking or chemical coupling) or recombinant techniques (e.g., fusion proteins). Ligation of the protein drug to a heavily glycosylated K07 peptide can provide an extended serum retention time due to the increased hydrodynamic radius of the protein drug. In this description, such a ligated protein drug may be referred to generically as a “recombinant protein drug,” regardless whether the K07 peptide (or its analog) is ligated to the parent drug using recombinant technologies.

In accordance with embodiments of the invention, the protein drugs may be any protein drugs, such as hormones (e.g., insulin), cytokines, or antibodies or binding fragments thereof. In accordance with embodiments of the invention, antibodies may include monoclonal antibodies (mAbs), bispecific antibodies, diabodies, or antibody-drug conjugates. The following description will use bispecific antibodies to illustrate embodiments of the invention. Again, this is for clarity of illustration and one skilled in the art would appreciate that other modifications and variations are possible without departing from the scope of the invention.

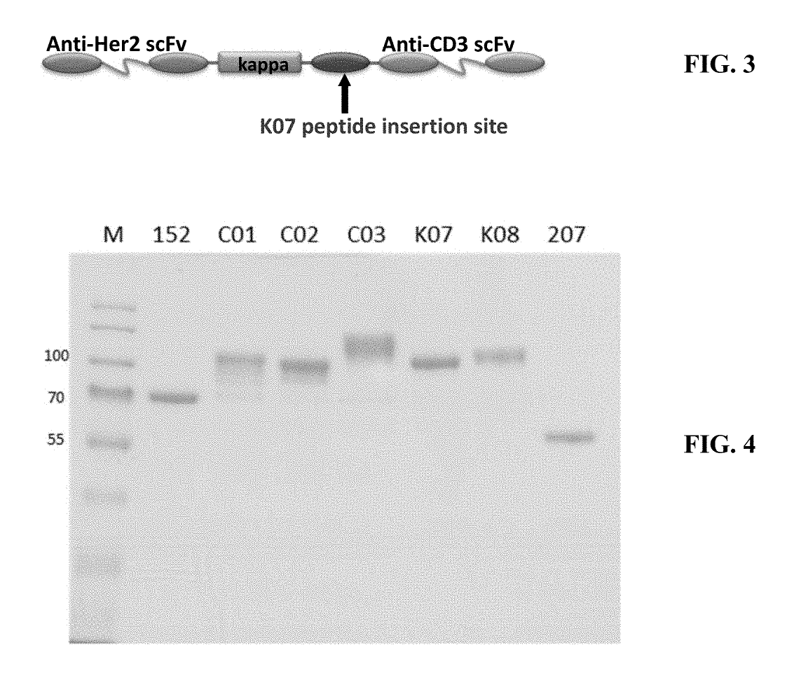

A bispecific antibody (bsAb) may comprise two specific binding domains (e.g., variable domains or scFv) linked by a bridging domain. An example of a bsAb may comprise an anti-Her2 scFv and an anti-CD3 scFv joined together by a kappa light chain and linker (FIG. 3). Ligation of the scFv bsAb (#152) to the heavily glycosylated K07 peptide can extend the serum retention time.

The present invention involving the K07 peptide may be applicable to not only antibody fragments such as single chain fragment variables (scFvs), but also small peptide drugs and therapeutic proteins. The results from PK studies presented in the later sections of this description indicate general applicabilities of the K07 peptide or similar peptides to enhance the serum half-lives of various peptides or proteins.

Embodiments of the invention will be further illustrated with the following examples. One skilled in the art would appreciate that these examples are for illustration only and that other modifications and variations are possible without departing from the scope of the invention.

### Example 1: Identification of highly glycosylated regions in human kininogen-1 protein

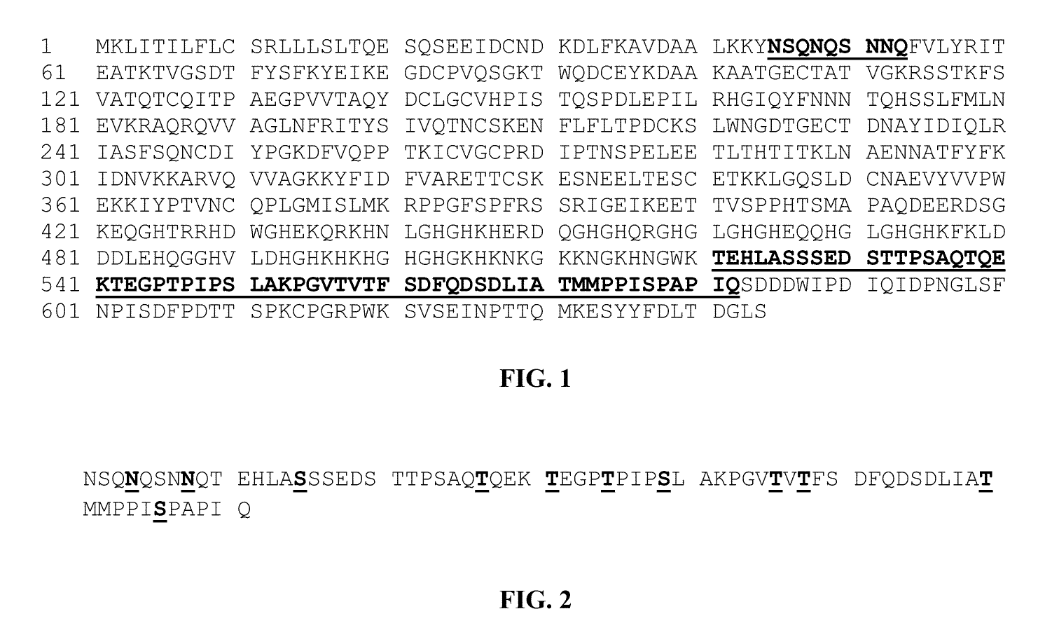

Human kininogen-1 protein (FIG. 1; SEQ ID NO:1) is a protein of 644 amino acids. Using BLAST searches and analysis, two highly glycosylated regions were found. These two regions are highlighted with bold face and underline in FIG. 1. In accordance with one embodiment of the invention, these two peptides are spliced together to generate a 71 amino acids long artificial peptide, NSQNQSNNQT EHLASSSEDS TTPSAQTQEK TEGPTPIPSL AKPGVTVTFS DFQDSDLIAT MMPPISPAPIQ (SEQ ID NO:2), referred to as K07 in this description (FIG. 2).

This K07 peptide contains nine potential 0-linked and two N-linked oligosaccharide attachment sites. This K07 peptide is expected to retain the glycosylation potentials. Therefore, attachment of this peptide or its derivative or analogs to a peptide or protein drug should increase the hydrodynamic radius of the peptide or protein drug.

### Example 2: Construction of K07-containing bsAb vector

To illustrate the utility of K07 peptide in extending the in vivo half-lives of protein drugs, we constructed fusion proteins using the K07 peptide. For example, an expression plasmid of a bispecific antibody #152 (bsAb #152) was prepared following procedures described in the U.S. Patent Application Publication No. 2013/0165638 A1, “LIGHT CHAIN-BRIDGED BISPECIFIC ANTIBODY.” Briefly, DNA digestion was performed in appropriate reaction buffers with BglII and BamHI restriction enzymes (New England BioLabs, Ipswich, Mass., USA) at concentrations of 1-10 units/mg of plasmid DNA for 1-3 hr at 37° C. The completed reaction was confirmed by agarose gel electrophoresis. Excised bsAb-encoded DNA fragments were then extracted from agarose by Agarose Gel Extraction kit (New England Biolabs) and inserted into BglII/BamHI cutting sites of the eukaryotic expression vector pTCAE8.3 to generate the bsAb #152 plasmid. Further modifications may be introduced by additional PCR and subcloning steps.

The bsAb construct used as a parent protein drug in this example is designated as bsAb #152 (FIG. 3). It has an anti-Her2 scFv (the first target domain) at the N-terminus and an anti-CD3 scFv (the second target domain) at the C-terminus joined together by a kappa light chain constant region (the bridging domain) and a short inter-domain linker (GGGGSGGGGSGGGGS; SEQ ID NO:4).

In accordance with one embodiment of the invention, a kininogen-1 (KNG1) derived peptide may be inserted at the site between the kappa bridge and the inter-domain linker through cloning, as illustrated in FIG. 3. Alternatively, the KNG1 derived peptide may be inserted at other locations, such as between the first target domain and the kappa bridge, or between the inter-domain linker and the second target domain. Similarly, the KNG1 derived peptide (e.g., K07) may be fused at the N-terminus or the C-terminus of the bispecific antibody. One skilled in the art would appreciate that the KNG1 peptide is to increase the hydrodynamic radius of the resulting protein, thereby increasing the in vivo half-lives of the protein drugs. Therefore, the fusion or insertion location of this peptide is not critical and one skilled in the art can easily test out several alternatives to optimize the desired results.

### Example 3: Expression and Purification of K07-Containing bsAb

After construction of the bsAb #152 DNA plasmid with the K07 insertion (kininogen-1 peptide-encoding DNA insert), it was transfected into the FreeStyle 293 cell line for transient expression. Cells were grown in Gibco FreeStyle 293 Expression Medium (Thermo Fisher Scientific) for 7 days and then centrifuged to remove the cell pellet and debris. The supernatant containing the antibody was passed through a 0.22 μm filter and then loaded onto a KappaSelect chromatography column (GE Healthcare Life Sciences) to isolate and purify the antibody. Purified bsAb was further concentrated by Amicon filter (Merck Millipore, Darmstadt, Germany) and buffer exchanged into PBS for storage at 4° C. Antibody concentration was confirmed by the BCA assay and analyzed by SDS-PAGE (FIG. 4). Antibody purification by the KappaSelect chromatography column yielded protein purity of over 90%.

In FIG. 4, Lane 2 (label 152) is the parental bsAb construct #152 and lane 6 (label K07) with a higher molecular weight (Mw) is the purified K07-bsAb construct with the K07 peptide insertion. Because the theoretical Mw increase contributed by K07 peptide sequence is only 7.5 kDa, the dramatic difference in Mw between these two bands on an SDS-PAGE gel indicates that the K07 peptide insert is heavily glycosylated.

### Example 4: Binding Assay

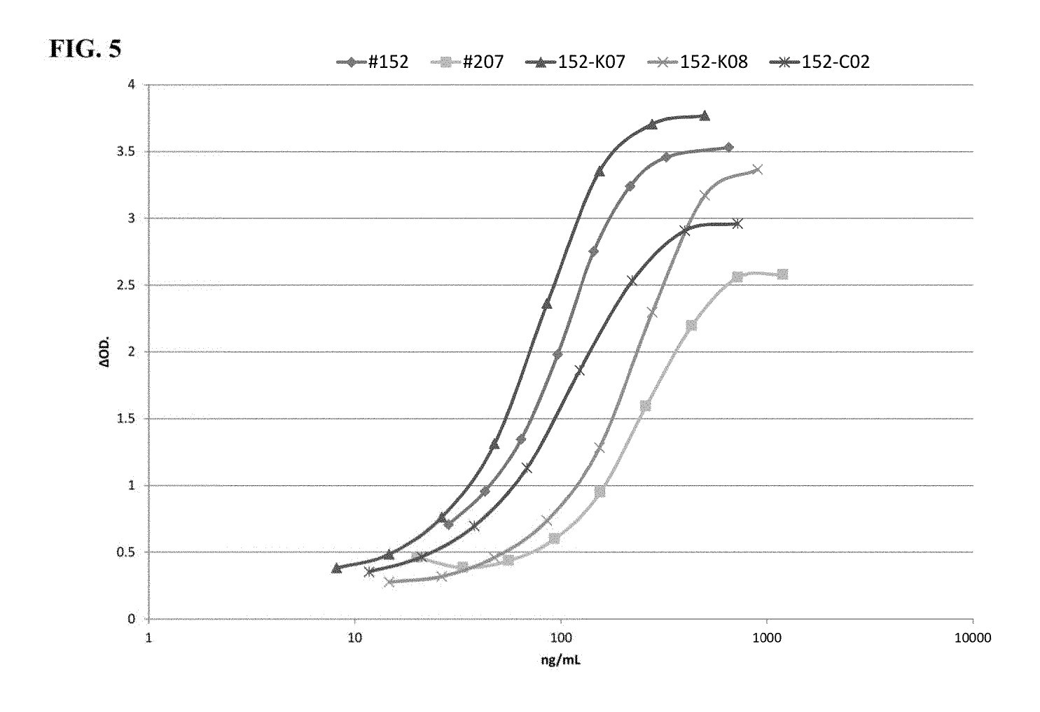

To investigate whether attachment of the K07 peptide impacts the activity of bsAb, in vitro functional assays to measure bsAb antibody domain affinity for the Her2 antigen were performed with enzyme-linked immunosorbent assays (ELISA) as shown in FIG. 5. Establishment of a linear range within the sigmoidal curve would be necessary to quantitate the bsAb antibody concentration of the mouse serums collected from the PK time points. Standard curves were first generated for both the parental bsAb construct #152 and the parental bsAb with K07 peptide insertion (#152-K07).

Recombinant human Her-2 protein (Bander Medsystems) encoding amino acids 23-652 was diluted in coating buffer (0.1 M, pH 9.6) to 1 μg/ml for use in coating of 96-well MaxiSorp plates (Nunc Inc., Roskilde, Denmark). The plates were incubated with protein at 4° C. overnight in moisture chamber. After washing three times with PBST (PBS with 0.05% Tween-20) buffer, the wells were blocked with 100 μl of blocking buffer (1% BSA in PBS) and incubated at 37° C. for 1 hr. After three more washes of PBST, the Her-2 antigen coated wells were then incubated with 100 μl of two-fold serial diluted K07-containing bsAb and incubated at 37° C. for 1 hr. This was also followed by three washes with PB ST. Goat anti-human kappa light chains HRP conjugated antibody (Sigma) diluted with blocking buffer was added to each well (100 μl/well) and incubated at room temperature for 1 hr, followed by washing 3 times with PB ST. Finally, 100 μl of TMB substrate (BioRad) were added to each well and incubated according to instructions. The reaction was stopped by adding 100 μl of 1.0 N HCl. BsAb binding affinity was measured at dual absorbance of 450 and 650 nm.

The results of the ELISA assays show that the parental bsAb #152 and bsAb #152-K07 have robust binding affinities for the Her2 antigen and were able to be saturated at an OD range between 3.5 and 3.75.

These results indicate that attachment of the highly-glycosylated peptide not only did not interfere with antibody bindings, but actually enhanced the binding, as manifested in the left shift (to the lower dissociation constant) of the binding sigmoid curve. This finding is unexpected.

### Example 5: Pharmacokinetics (PK) Studies

We next investigated the pharmacokinetics (PK) of the modified bsAb. In the PK study, the control antibody is bsAb #152, which is without the K07 peptide insert. These antibodies were injected into mice. Serum samples were collected from mice before the injection (pre-dose) and at 11 time points after the injection of the bsAb samples. Serum concentrations of the bsAbs were determined with ELISA and shown as concentration (μg/mL) vs. time (hours).

In the PK study, BALB/c male mice at 8 weeks old were injected with the testing bsAbs via tail vein at an antibody concentration of 3.0 mg/kg. PK blood samples were collected at time points of 0 min (pre-dose), 5 min, 15 min, 30 min, 1 hr, 2 hr, 4 hr, 8 hr, 24 hr, 48 hr, 96 hr, and 192 hr. Three mice were in each group and about 30 μL of blood sample volume was taken at each time point. The serum was collected after the blood was clotted and centrifuged. Collected mouse serum samples were kept at −70° C. until quantitative bio-analysis by ELISA for antibody concentration determination.

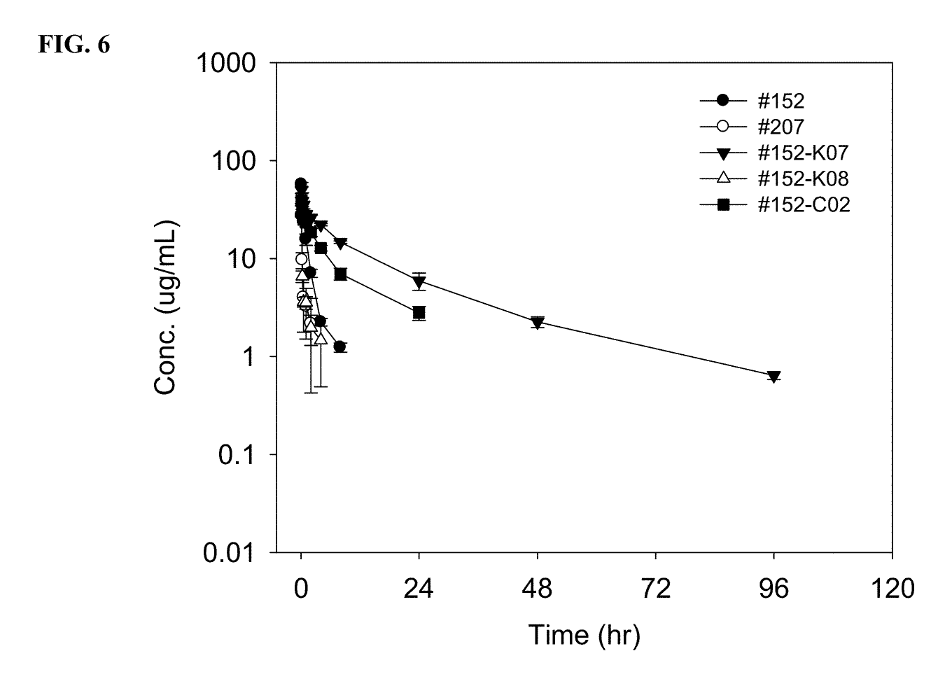

As shown in FIG. 6, the bsAb #152 with or without the K07 peptide insert showed similar retention ability (Cₘₐₓ) in mouse serum after initial injection. However, the parental bsAb #152 concentrations rapidly decreased in mouse serum. The K07-containing bsAb (#152-K07), in contrast, retained higher concentrations in the serum for a longer duration after the injection. For a drug, the relatively fast decay in the blood would decrease the effective concentration too quickly and would require more frequent administrations. The longer in vivo half-life indicates that the K07-containing drug would have much improved pharmacokinetic behavior and would not require frequent administrations.

### Example 6: Confirmation of High Glycosylation Potentials in K07 Peptide

To understand the glycosylation status at the N- and O-linked glycosylation sites of K07-containing bsAb, especially in the K07 peptide insert, mass spectrum analyses were performed. All the glycosylation sites are identified by MS/MS spectrum with different dissociation methods.

The bsAb #152-K07 fusion protein was generated and purified from the Freestyle 293 cell line. 100 μg of the protein was treated with 10 mM DTT at 80° C. for 15 mins and alkylated with 55 mM iodoacetic acid at R.T. for 30 mins. The reduced protein was diluted to a final concentration of 1 μg/μl in 100 μl of 50 mM ammonia bicarbonate and digested with 1 μg protease K overnight. The samples were diluted with formic acid to 0.1% and send to mass analysis.

For LC-MS spectrum analysis, samples were subjected to Waters UPLC H-class Biosystem with the separation column BEH C18 (2.1 mm×150 mm, 1.7 μm). The composition of solution A and B were 0.1% formic acid in water and acetonitrile, respectively. The mobile phase was increased from 5 to 95% of solution B in 100 minutes. Separated samples from UPLC were connected directly to the ESI-MS instrument, Synapt G2-Si (Waters Inc. Milford, Mass. USA). The data were collected by way of 0.5 second MS followed with data dependent acquisition (top 5 method) and the MS fragmentations were generated with either collision-induced dissociation (CID) or electron-transfer dissociation (ETD). Data were processed by Unifi™ (V1.8) including the FASTA format data bank focused on sequences of antibodies.

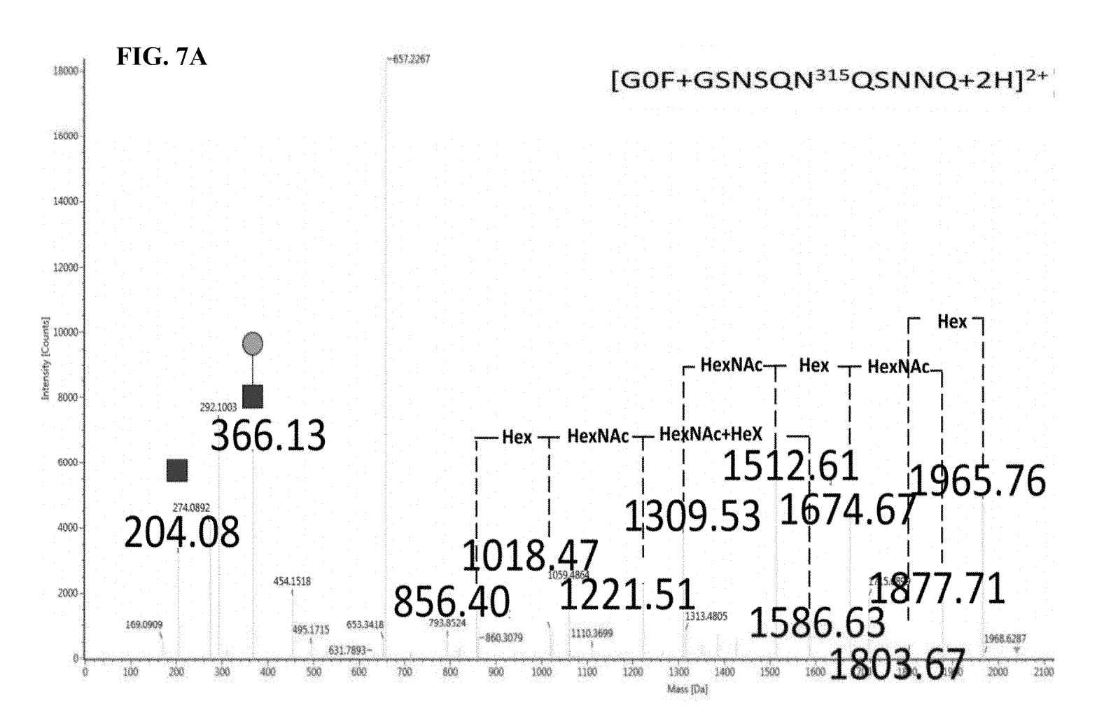

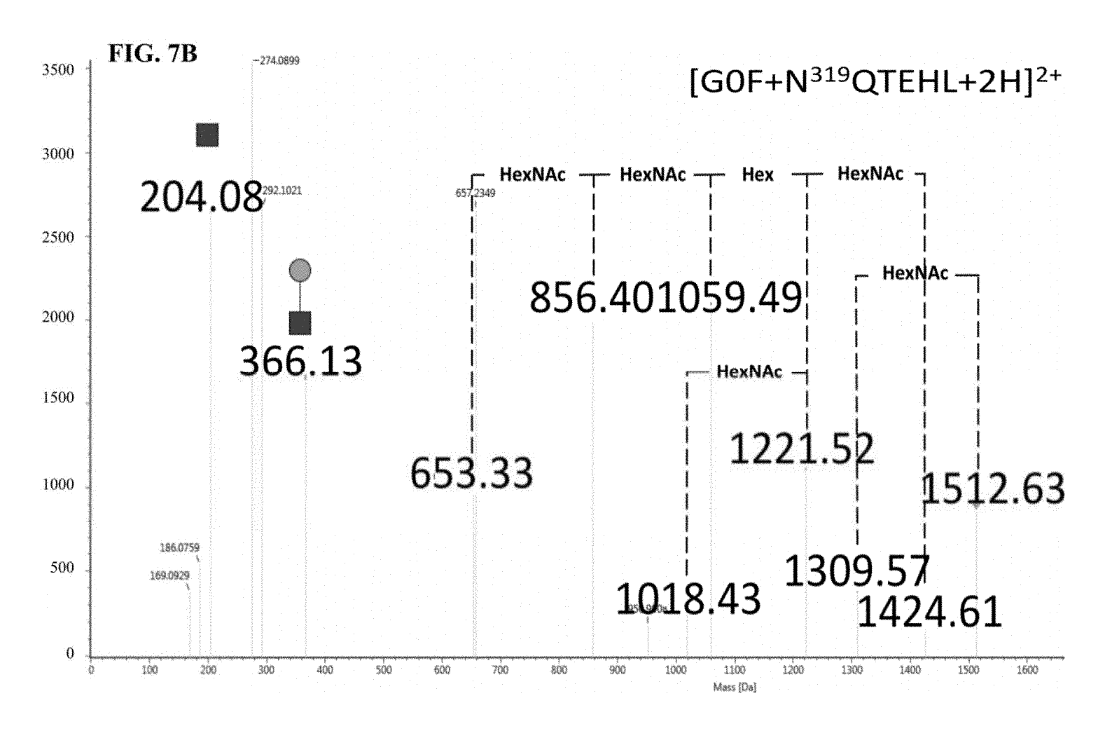

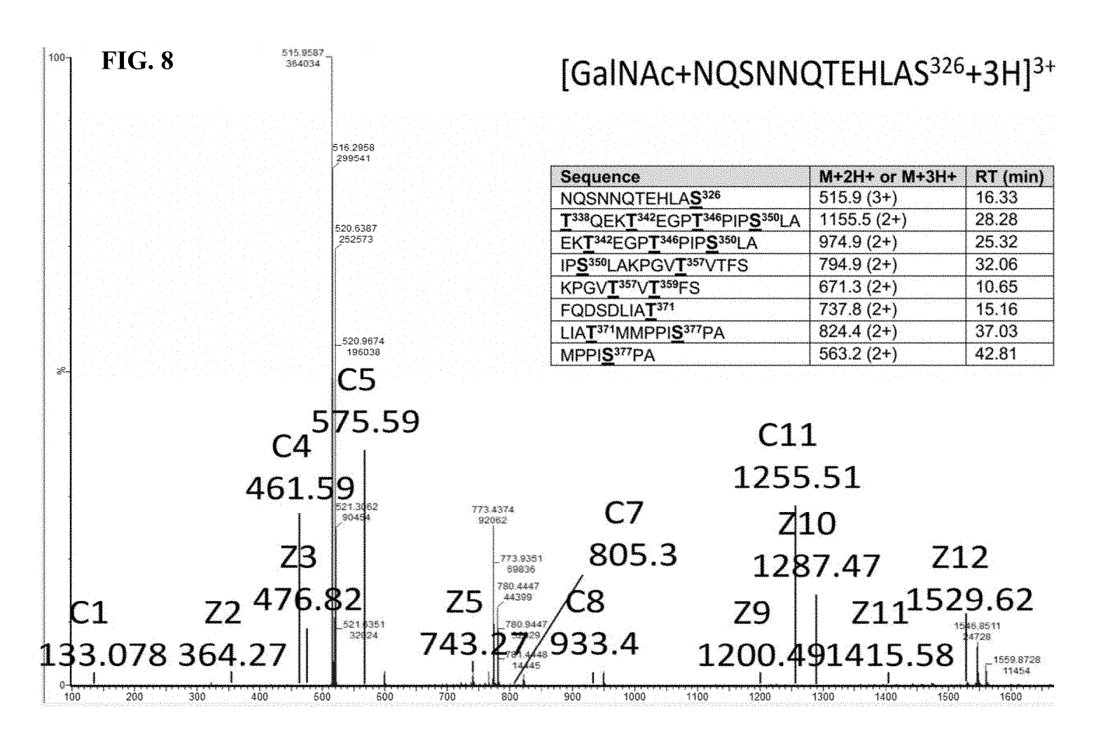

After completion of data acquisition in mass analysis, the components that have been identified to be a glycosylated peptide are calculated according to the predicted peptide mass and signature ions from oligosaccharides. The N glycosylation sites were identified at N³¹⁵ and N³¹⁹ (the numbers are based on the fusion protein sequence; these two residues correspond to N⁴ and N⁸ in the sequence shown in FIG. 2) with CID annotation and GOF glycoforms on these sites (FIGS. 7A and 7B). The O glycosylation sites were identified by Unifi software and ETD MS/MS. The ETD spectra contained enough c and z ion peaks to locate all the glycosylation sites. Six threonine residues at positions 338, 342, 346, 357, 359, and 371 as well as three serine residues at positions 326, 350, and 377 are sequenced and assigned (FIG. 8, highlighted as bold and underlined). One representative ETD spectrum annotation was presented in FIG. 8 (top panel). The series of C and Z ions were labeled and showed the GalNac ion is located at AS site. As the O glycosylation will only occur on serine, threonine, and tyrosine sites, the S³²⁶ is the glycosylated site.

Collectively, mass spectrum analyses indicate a KNG1 derived peptide (e.g., K07) can be highly glycosylated when it is fused to a peptide or protein drug (e.g., bsAb) and expressed in a eukaryotic cell line (e.g., FreeStyle 293). One skilled in the art would appreciate that the KNG1 peptide is to retain the high glycosylation potentials in the resulting recombinant proteins, thereby increasing the hydrodynamic radius and the in vivo half-lives of the protein drugs. Therefore, the number of glycan linkage site and the glycan composition of this peptide are not critical and one skilled in the art can easily test out several alternatives to optimize the desired results. Such alternatives, for example, may include repeating or deleting some of the glycosylation sites shown in K07 to obtain analogs of K07 peptides, which would confer similar properties.

### Example 7: Glycosylation Extent of K07 Influences Its Pharmacokinetics (PK)

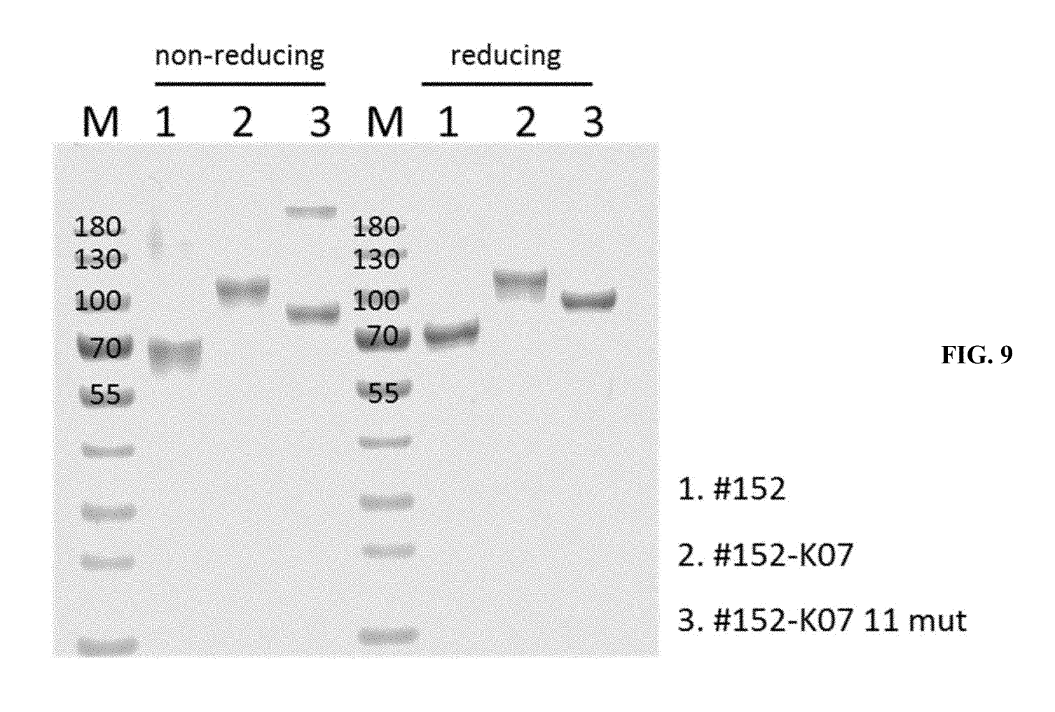

To further illustrate the heavily-glycosylated K07 peptide in extending the in vivo half-lives of protein drugs, we mutated multiple glycosylation sites in the K07 peptide insert, reduced the glycosylation extent of K07-containing proteins, and subjected resulting proteins to a pharmacokinetics (PK) study. For example, the bsAb #152-K07 11 mut is a mutant form derived from the bsAb #152-K07 by substituting alanine for eleven amino acid residues (the K07 11 mut is SEQ ID NO:3) in the K07 peptide insert. The purified proteins of the parental bsAb #152, the bsAb #152 with the K07 peptide insert (#152-K07), and the bsAb #152-K07 with eleven mutations (#152-K07 11 mut) were examined in SDS-PAGE (FIG. 9). As shown in FIG. 9, the lower molecular weight (Mw) of #152-K07 11 mut when compared with #152-K07 indicates a dramatic reduction in the glycosylation extent within the K07 peptide region. However, the still significant higher Mw of #152-K07 11 mut when compared with #152 suggests other (less potent) glycosylation sites may exist and become glycosylated when eleven mutations were introduced into the K07 peptide insert.

In the PK study, BALB/c male mice at 8 weeks old were injected with the testing bsAbs via tail vein at an antibody concentration of 3.0 mg/kg. PK blood samples were collected at time points of 0 min (pre-dose), 5 min, 15 min, 30 min, 1 hr, 2 hr, 4 hr, 8 hr, 24 hr, 48 hr, 96 hr, and 192 hr. Six mice were in each group and about 30 μL of blood sample volume was taken at each time point. The serum was collected after the blood was clotted and centrifuged. Collected mouse serum samples were kept at −70° C. until quantitative bio-analysis by ELISA for antibody concentration determination.

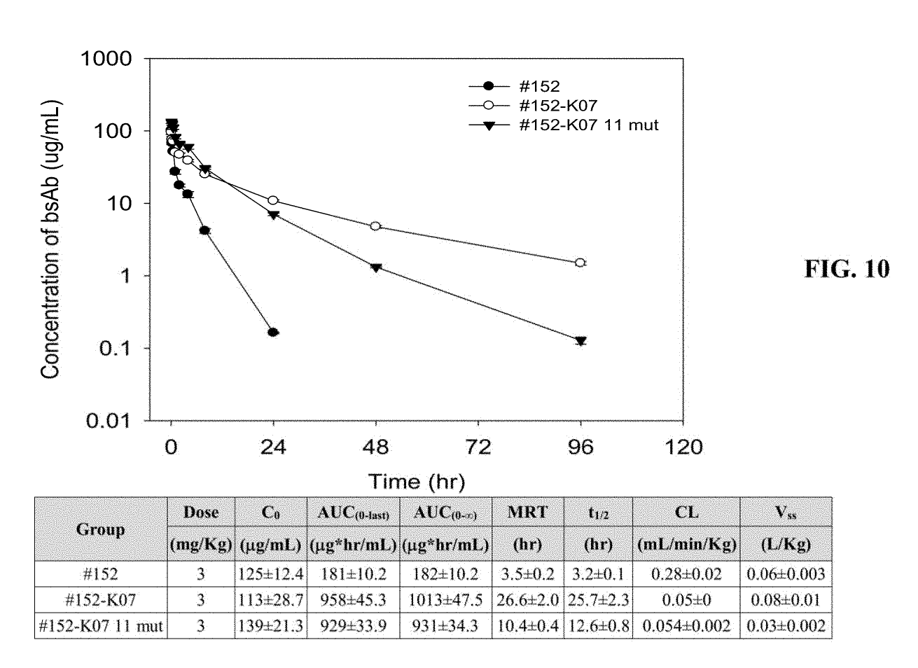

As shown in FIG. 10, the bsAb #152 in the presence or absence of the K07 peptide insert (with or without mutations at the glycosylation sites) showed similar retention ability (Cmax) in mouse serum right after initial injection. Like the results of FIG. 6, the concentrations of the parental bsAb #152 decreased rapidly and the concentrations of the #152-K07 retained higher in the serum for a longer duration after the injection. Interestingly, the concentrations of the #152-K07 11 mut decreased faster than those of the #152-K07 but slower than those of the #152, although the #152-K07 11 mut showed the highest concentrations at earlier time points (<8 hr). The half-life (tin) of the parental bsAb #152 increased eight-fold (from 3.2±0.1 hr to 25.7±2.3 hr) when the K07 peptide was inserted; however, only four-fold increase (from 3.2±0.1 hr to 12.6±0.8 hr) was observed when the mutant K07 peptide was inserted. From these data, one can conclude that the in vivo half-life extensions of recombinant proteins are attributable to the heavily-glycosylated K07 peptide.

It is important to note that even with 11 amino acid substitutions at the glycosylation sites in the 71-amino acid K07 peptide, the resultant peptide still can extend the in vivo half-lives of proteins. This represents a 15% (11/71) mutation in the K07 peptide and these mutations involve important residues (i.e., glycosylation sites). One skilled in the art would appreciate that further inclusion of non-critical residue mutations (e.g., substitutions with homologous residues) can certainly be tolerated. Therefore, it is reasonable to use an analog of K07 having a sequence identity of 80% or higher with SEQ ID NO:2, preferably 85% or higher, more preferably 90% or higher, most preferably 95% or higher, in embodiments of the invention. These mutants of K07 peptides, which can still confer the properties of extended in vivo half-lives to a protein drug, will be referred to as “K07 homologs” or “homolog” of K07 peptide. These homologs may be further defined by their extent of identity to the K07 sequence (SEQ ID NO:2) set forth in FIG. 2, e.g., a homolog of K07 with 80% sequence identity or a homolog of K07 with 90% sequence identity. Because such peptides or homologs are derived from kininogen-1, they may also be referred to generically as “modified kininogen-1 peptides.”

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A recombinant protein drug, comprising: a parent protein drug coupled with a modified kininogen-1 peptide.

2. The recombinant protein drug according to claim 1, wherein the modified kininogen-1 peptide has 80% or higher sequence identity with SEQ ID NO:2.

3. The recombinant protein drug according to claim 1, wherein the modified kininogen-1 peptide has 85% or higher sequence identity with SEQ ID NO:2.

4. The recombinant protein drug according to claim 1, wherein the modified kininogen-1 peptide has the sequence of SEQ ID NO:2.

5. The recombinant protein drug according to claim 1, wherein the parent protein drug is a bispecific antibody having a first targeting domain linked by a bridging domain with a second targeting domain.

6. The recombinant protein drug according to claim 5, wherein the modified kininogen-1 peptide is fused between the first targeting domain and the bridging domain, or between the bridging domain and the second targeting domain.

7. A method for increasing serum half-life of a protein drug, comprising: attaching a modified kininogen-1 peptide to the protein drug.

8. The method according to claim 7, wherein the modified kininogen-1 peptide and the protein drug form a fusion protein.

9. The method according to claim 7, wherein the modified kininogen-1 peptide has 80% or higher sequence identity with the sequence of SEQ ID NO:2.

10. The method according to claim 7, wherein the modified kininogen-1 peptide has the sequence of SEQ ID NO:2.

11. The method according to claim 7, wherein the protein drug is a bispecific antibody having a first targeting domain linked by a bridging domain with a second targeting domain.

12. The method according to claim 11, wherein the modified kininogen-1 peptide is fused between the first targeting domain and the bridging domain, or between the bridging domain and the second targeting domain.

13. The recombinant protein drug according to claim 2, wherein the parent protein drug is a bispecific antibody having a first targeting domain linked by a bridging domain with a second targeting domain.

14. The recombinant protein drug according to claim 3, wherein the parent protein drug is a bispecific antibody having a first targeting domain linked by a bridging domain with a second targeting domain.

15. The recombinant protein drug according to claim 4, wherein the parent protein drug is a bispecific antibody having a first targeting domain linked by a bridging domain with a second targeting domain.


SEQUENCE LISTING (4 sequences)

SEQ ID NO: 1 - protein, 644 aa, Homo sapiens
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 2 - protein, 71 aa, Artificial Sequence
  Synthetic
NSQNQSNNQTEHLASSSEDSTTPSAQTQEKTEGPTPIPSLAKPGVTVTFSDFQDSDLIATMMPPISPAPIQ

SEQ ID NO: 3 - protein, 71 aa, Artificial Sequence
  Synthetic
NSQAQSNAQTEHLAASSEDSTTPSAQAQEKAEGPTPIPALAKPGVAVAFSDFQDSDLIAAMMPPIAPAPIQ

SEQ ID NO: 4 - protein, 15 aa, Artificial Sequence
  Synthetic
GGGGSGGGGSGGGGS